(19) United States
(12) Patent Application Publication
(10) Pub. No.: US 2007/0118016 A1
(43) Pub. Date: May 24, 2007

(54) Endoskop, insbesondere duodenoskop fur die mutter-baby-cholangioskopie

(76) Inventors: Eckart Frimberger, Muenchen (DE); Viktor Josef Wimmer, Seeon (DE)

Correspondence Address: ST. ONGE STEWARD JOHNSTON & REENS, LLC, 986 BEDFORD STREET, STAMFORD, CT, 06905-5619, US

(21) Appl. No.: 11/507,125

(22) Filed: Aug. 21, 2006

(30) Foreign Application Priority Data
Aug. 19, 2005 (DE) 10 2005 039 601.1

(51) Int. Cl. A61B 1/00

(52) U.S. Cl. 600/154; 600/153

(57) ABSTRACT

An endoscope, in particular a duodenoscope, for mother-baby cholangioscopy, having a working canal that runs in or on a shaft of the endoscope and includes an outlet opening distally on the endoscope head, so that the working canal includes at least a first insertion opening, which lies in an area of the shaft far from the proximal end of the endoscope.

### FIELD OF THE INVENTION

Endoscopes of this type find numerous applications in endoscopy. Usually, in this area, an instrument, such as a catheter, a small so-called baby endoscope with a small diameter, or the like, is introduced within an instrument canal running from the proximal to the distal endoscope end of an already placed large endoscope, in order to conduct an endoscopic examination or a procedure.

### BACKGROUND OF THE INVENTION

Thus, for instance, in peroral transpapillary cholangioscopy, first a flexible mother endoscope (large duodenoscope), following the usual ERCP technique, is placed on the papilla major (hereafter referred to as papilla). To reach the papilla, a large portion of the duodenoscope shaft must be inserted into the patient (gullet, alimentary canal, stomach, duodenum), because the endoscope must be pushed along the reverse-curvature of the stomach into the duodenum. This is followed by a stretch maneuver, in which a large portion of the shaft lengths is extracted, until finally the duodenoscope lies extended to the papilla. After the extension, only about 60 cm more of the front endoscope shaft is introduced into the patient. In this extended position, both ERCP and mother-baby cholangioscopy are conducted.

The heretofore usual standard mother-baby cholangioscopy demands a (large) jumbo or mother duodenoscope and a baby cholangioscope that is introduced into the mother duodenoscope. The baby endoscope here is introduced into the gall passages by way of an opening of the instrumental or working canal of the mother endoscope that is found on the endoscope housing or operating part. The method serves primarily to fragment large gallstones, for example with a laser probe, and to diagnose unclear gall bladder findings.

The disadvantage with this known instrumentation and method is that the baby endoscope is difficult or nearly impossible to maneuver. In addition, many years' practice with frequent applications reveals a high need for repairs of the baby endoscope, which frequently becomes defective even after a few uses.

### SUMMARY OF THE INVENTION

It is therefore the object of the present invention to improve this state of the art and to create an endoscope, as well as a device made up of a mother and baby endoscope, which will avoid these disadvantages and ensure improved maneuverability along with less frequent need for repair.

The invention fulfills the objective by means of an endoscope with the characteristics of claim 1, a baby endoscope with the characteristics of claim 10, and a device made up of such an endoscope with a baby endoscope of the same type.

Because the first entry opening, hereafter referred to as the shaft port, is located in an area at a distance from the proximal end of the endoscope, the shaft port after a successful extension lies outside the patient, for instance a few centimeters before the mouth, and can now be used as an entry into the instrument canal or working canal of the endoscope. In this manner a considerably shortened baby endoscope can be used, because it is inserted through the shaft port and no longer through the otherwise usual (proximal) entry or the proximal port of the instrument canal on the operating part of the endoscope. Because of instruments, such as a babyscope, a catheter, etc., which require a length of about 200 cm for instance with a diameter of 3 to 4 mm extending beyond the shaft length of the mother endoscope, it is now possible advantageously to use considerably shortened instruments, for instance a baby endoscope with a length of only 90 to 140 cm, in particular 100 to 120 cm.

The shortened instrument, in particular the shortened baby endoscope, is more maneuverable, can turn better than traditional longer instruments, and moreover the cleaning of such a shorter instrument becomes easier. In addition its ability to turn corners is also improved, and there is also the cost advantage of reduced need for repair.

Short instruments for consideration here, in addition to a baby endoscope (cholangioscope, pancreatoscope), also include for instance catheters of the angiography catheter type that resist turning and are inserted into the gall passages by way of the first entry opening or the shaft port in order to reach gall branches previously having only very limited accessibility or none at all. While angiography catheters, which are inserted by normal duodenoscopes, can no longer turn because of their great length, this is now possible because of the shortened extension of the shaft port, or its shortened configuration. Thus it is now possible to make precise soundings of the various segments of the tree-like biliary system, which can be reached only by long, patient “poking” or vague probing by guide wires and catheters, more or less by chance or not at all.

In an advantageous configuration of the invention, the shaft port lies in the central area of the shaft, in particular at a distance of 60 to 70 cm from the distal end of the endoscope. This makes it possible to ensure, in particular examinations, especially those of cholangioscopy, that the first opening comes to lie outside the patient, yet close to a bodily orifice, for instance the mouth. An instrument that is to be inserted therefore needs only to have a minimum length of, for instance, 80 to 140 cm, preferably 120 cm, in order to extend beyond the part of the shaft that is in the patient after placement.

In a preferred configuration of the invention the endoscope additionally has, in the area of the proximal end or on the endoscope housing, a further insertion opening in the form of a customary proximal port. As a result the endoscope can be used additionally as a normal endoscope, for instance a duodenoscope, sparing the costs for a separate acquisition of such an endoscope.

Consequently the first and the additional insertion opening or shaft port and the proximal port can lead to one and the same working canal, so that the working canal as a result extends all the way to the proximal port. As a result, advantageously, an additional, separate working canal can be avoided, which otherwise might possibly lead to an undesired increase in the shaft diameter of the endoscope.

In an additional configuration of the invention, the shaft port is configured as closable and insulated with a covering. This can be obtained, for instance, through a covering in the form of a cap that can be secured in stable position from the outside, which preferably insulates both against the working canal and from inside the endoscope when it is in place, for instance bolted. Other possibilities can also be considered, of course, for providing an insulating covering for the first insertion opening or the shaft port, especially during the phase of placing the endoscope. This can be achieved, for instance, by means of a probe inserted into the endoscope canal through the conventional entry on the operating part of the endoscope, in particular a hollow probe (to allow an additional insufflation), which provides an insulating cover for the shaft port from inside (inner covering) or by means of a closing sleeve, whose opening is closed by rotating or pushing with respect to the shaft port opening (external covering). It is also possible to use a thin-walled rubber or plastic hose (for instance of the “finger-cap” type with a cut-off tip), which is pushed down onto the duodenoscope and unrolled over the shaft port opening (external covering).

In an additional configuration of the invention, an addition can be placed on the shaft port with an insertion support so that it is firmly in place and removable, in order to facilitate the insertion of instruments into the working canal. This port addition preferably insulates the first insertion opening or the shaft port, so that, possibly by using a sealing cap or insulation, no air can escape. The addition in this case can be positioned in such a way on the shaft port or in this area of the shaft that the insertion support comes to rest, laterally displaced in its cross-section profile, for instance by 90 to 180 degrees, on the other side of a customary proximal port. As a result, advantageously, it is possible to avoid having operating personnel and the physician get in one another's way during a procedure, because now the opposite side (that is, looking leftward from the operating part, for instance) of the usually proximal port (in the rule, right-sided) is used for various tasks or for inserting and operating an instrument.

In a preferred embodiment of the invention the area of the endoscope shaft, in which the shaft port is found, is configured as a short, for instance smaller than 10 cm, especially 4 to 6 cm long, rigid (shaft-) sleeve, for instance in the form of a rigid outer shaft segment and a likewise rigid inner canal segment. As a result, despite good flexibility of the total endoscope shaft, there is an advantageous increase in the stability and precise fit of the shaft port, so that on this a covering in the form of a mountable cap or a port addition at the flattest possible angle of insertion can be positioned that is insulating and stationary.

Additional advantageous configurations of the invention can be seen from the subsidiary claims.

The invention is explained in greater detail hereafter with reference to the embodiments depicted in the illustrations.

### BRIEF DESCRIPTION OF THE DRAWINGS

FIG. 1a shows a side view of a shaft port of an endoscope according to the invention, with cap and working canal.

FIG. 1b shows a cut-out view of the shaft port according to FIG. 1a along the line A-A.

FIG. 1c shows a cut-out view of the shaft port according to FIG. 1a along the line B-B.

FIG. 2a shows a view of a cap from below.

FIG. 2b shows a cut-out view of the cap according to FIG. 2a along the line C-C.

FIG. 3 shows an exploded view of a shaft port according to FIG. 1a.

FIG. 4a shows a side view of the shaft port with port addition.

FIG. 4b shows a cut-out view of the shaft port according to FIG. 4a along the line E-E.

FIG. 4c shows a cut-out view of the shaft port according to FIG. 4a along the line D-D.

FIG. 5 shows a side view of an endoscope according to the invention.

FIG. 6 shows a schematic cross-section through the endoscope according to FIG. 5 along the line F-F.

### DETAILED DESCRIPTION OF THE INVENTION

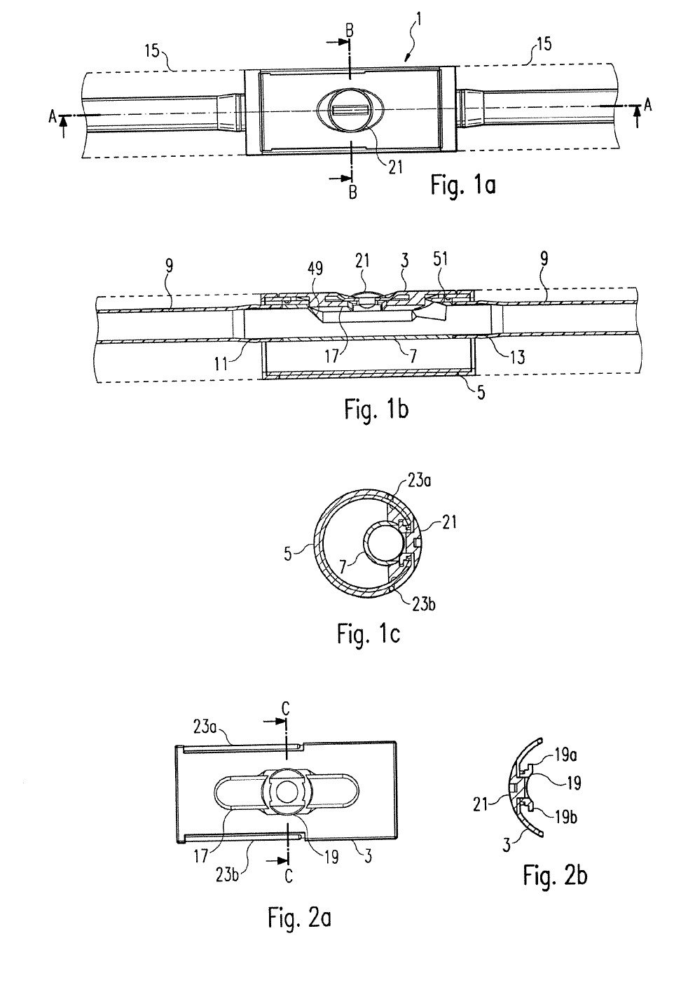

The first entry opening or the shaft port 1 of an endoscope, depicted in FIGS. 1a through 1c, is shown with an (external) covering in the form of a cap 3. The shaft port 1 includes a shaft segment in the form of an external rigid sleeve 5, as well as a canal segment in the form of a rigid sleeve 7 that is of lesser diameter, which serves as passage for a working canal 9 of the endoscope and is not concentric in cross-section (FIG. 1c) but rather is positioned close to the inner walling of the shaft segment 5.

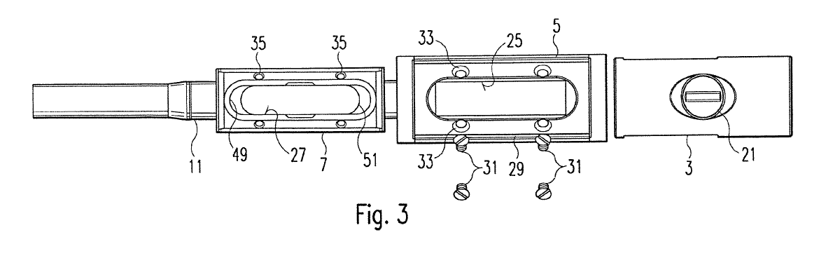

To make an opening that extends into the working canal 9, both the shaft segment 5 and the canal segment 7 preferably have lengthwise openings 25, 27 (FIG. 3) that are flush with one another. The access to the working canal 9 achieved in this way, as illustrated in FIG. 1b, can include an insertion aid in the form of a fiber 49 that widens toward the exterior in order to facilitate insertion of an instrument into the working canal 9. In addition, on the opposite short side of the length-wise shaft port opening, there can be a slope or insertion aid 51 that widens toward the inside, in order to facilitate the diagonal insertion of an instrument from diagonally left above into the working canal 9 leading to the right in the direction of the distal end. In this way, advantageously, even with a short shaft port 1 or a first insertion opening of for instance 15 to 30 cm, easy insertion of an instrument can be guaranteed.

For a bilateral connection to the working canal 9 configured as a Teflon hose, the canal segment 7 includes two connecting supports 11 and 13, on which the ends of the working canal 9 can be mounted and possibly, in addition, can be fixed in place, for instance by cementing.

The shaft segment 5 is firmly, for instance by cementing, connected flush in the endoscope shaft 1, which in FIG. 1a and FIG. 1b is shown with broken lines, so that no undesired gripping points, like steps, edges, or the like, are formed as obstacles to sliding on the outer surface of the endoscope shaft 15, despite the preferably rigid configuration of the entire shaft port 1.

As can be seen from FIG. 1b, the thick cap 3 (with a width of 0.3 mm, for instance) shuts off the shaft port 1 or the first insertion opening by covering it, so that a protruding area 17 on the underside of the cap 3 extends into the lengthwise opening 25 of the shaft segment 5 and possibly even into the opening 27 of the canal segment 7 that is flush with it, preferably fitting exactly. As a result it can be guaranteed in simple manner that the cap 3 can be secured so that it is stationary on the shaft port 1. For this purpose an actuating element, for instance in the form of a slitted screw 21 of a bolting device, is actuated, so that the locking device consists for instance of a locking element 19. This locking element 19 is configured in oval shape on the underside of the bolt 21, so that only when it is locked do the opposite-facing widenings 19a and 19b of the essentially oval disc 19 engage in corresponding recesses of the canal segment 7. It is also possible, of course, to configure the cap 3 as flipping open, so that it is configured for this purpose in profile, for instance, as a half-cup extending beyond the half of the shaft circumference or surrounding it. The extending area 17 in any case can additionally be configured as flush to the canal interior, so that when it is locked, advantageously for an instrument inserted by the proximal port 63, no undesired resistances occur.

As can be seen from FIG. 3, the shaft port is constructed from a canal segment 7 and the shaft segment 5 in such a way that, as seen in FIG. 1c, the canal segment with it opening 27 is positioned on the inside of the shaft segment 5 in the area of the opening 25. For simple fastening, the canal segment 7 can have a semicircular widening in cross-section on the outer surface, in which threaded bore holes 35 are provided. When assembled, these bore holes 35 are flush with bore holes 33 in the shaft segment 5, so that the canal segment 7 can be fixed on the shaft segment 5 by means of screws 31. In this way the opening 25 of the shaft segment 5 with its slopes 49 and 51 and the opening 7 of the canal segment 7 lead flush into one another.

To ensure impenetrability for the endoscope shaft interior, the invention provides, between the outer surface of the canal segment 7, an insulation (not shown in detail in the illustration) that runs around the opening 27 and adheres to the inside of the shaft segment 5, insulating it. In addition, on the outer surface of the shaft segment 5, an insulation 29 is positioned running around the opening 25 in the area of the underside of the cap 3, so that the cap 3 when locked also closes and insulates. To increase counterpressure of the insulation 28 in closed position, one or more lengthwise spring elements 23a and 23b can be positioned in addition on both longitudinal sides of the cap 3 (or on the corresponding complementary recesses of the shaft port 1), and these spring elements are arched in such a way that they extend lengthwise during closing and their spring force works against the closing of the cap 3.

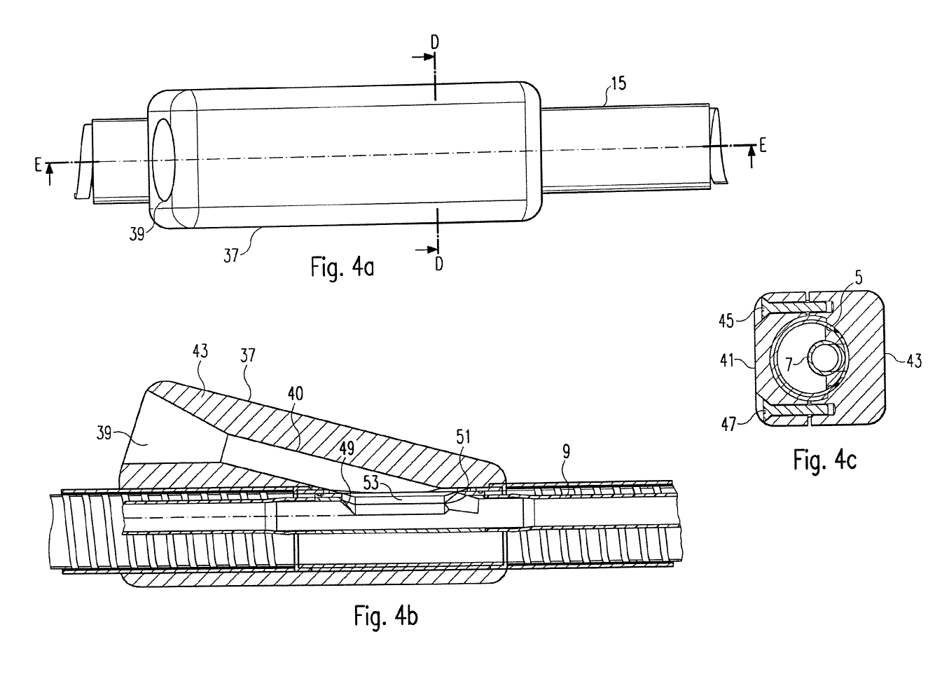

FIG. 4a shows how a port addition 37, which for instance is manufactured as a milled or die-molded part, is placed on the shaft port 1 to allow an instrument to be inserted through an opening 39 into the working canal 9 in the distal direction (toward the right in the illustration). The port addition 37 is thereby placed on the shaft port 1 of the mother endoscope shaft 15 through the lateral opening 25, 27. In addition, an insulation (O-ring, rubber plate, etc.), not shown in detail in the illustration, can be provided between the shaft port 1 and the port addition 37, to prevent air from escaping between the port addition 37 and the shaft port 1.

According to FIG. 4c, the port addition 37 is constructed of an underplate 41 and an overplate 43, in which are the funnel-shaped opening 37, widening outward, and the straight connecting insertion support 40. For placement on the shaft port 1, as illustrated, two securing elements 45 and 47 can be positioned, for instance in the form of locking pins or screws, though of course it is also possible to have a joint on one side and a lock positioned on the opposite side, for instance in the form of a hook.

As shown in FIG. 4b, the straight tubular-shaped insertion support 40 leads from left above to right diagonally below in a flat angle into the working canal 9 in the distal direction (right side in the illustration), so that this support is braced by slopes 49 and 51 connecting to one another on the short sides of the lengthwise openings 25 and 27. The insertion support 40 thereby has an inner diameter that is smaller than the inner diameter of the working canal (4.2 mm, for instance) or equal to it. In addition, the port addition at this location extends inward with its material 53 configured to correspond with the opening 25, so that because of this mutually fitting interlocking a fixed, non-rotatable positioning and centering of the port addition 51 on the shaft port is guaranteed. In this way, advantageously, it is possible to dispense with providing recesses or protuberances on the outside of the shaft 15 and/or of the shaft port 1, which are otherwise necessary for a stationary connection and which undesirably heighten the friction or sliding resistance during a procedure. Because of the insertion aids 49, 51, even with the desired short length of the shaft port or of the shaft and canal segments 5 and 7 of 40 to 60 mm and a short shaft port opening only 15 to 30 mm long, realized by the openings 15, 27, it is possible to have the flattest possible angle, or an acute angle (looking toward the proximal end), between the longitudinal axis of the insertion support and the longitudinal axis of the working canal 9 or of the endoscope shaft of, for instance, 10 to 40 degrees, preferably 10 to 20 degrees. In addition the insertion support 40 or the opening 39 can be closed with an insulating flap (also for insulated insertion of instruments), not shown in detail in the illustration.

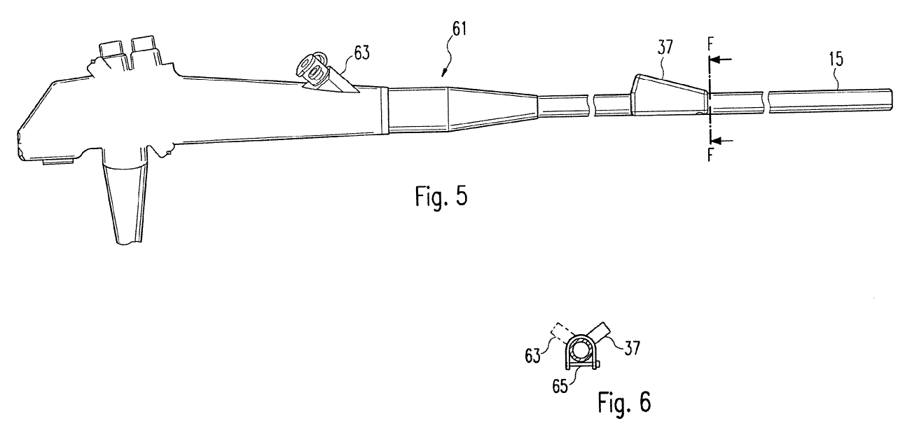

It can be seen from FIGS. 5 and 6 in what manner and in what position the shaft port 1 or the port addition 37 is positioned with a mother or mother duodenoscope 61 shown here. The shaft port 1, 40 to 60 mm long and integrated into the shaft, is found here in the longitudinal direction between about 60 and 70 cm distant from the distal head end of the endoscope 61. The lengthwise opening of the shaft port 1 has, for instance, for inserting a baby endoscope with a diameter of 3.5 mm a length of about 15 to 30 mm. Because of the port addition 37, the sharpest possible insertion angle becomes possible, for example about 10 to 30 degrees, to the longitudinal axis of the endoscope 61.

From the strongly schematized cross-sectional view according to FIG. 6 it is clear that the shaft port 1 or port addition 37, with its opening 39 in the longitudinal direction, is positioned on the proximal end or on the left side from the perspective of the operating part of the endoscope, whereas a conventional proximal port 63 is usually positioned on the operating part on the right side. Because of this opposite arrangement, for example of 90 to 180 degrees, in particular symmetrical to an arrangement on the middle axis, it is possible, advantageously, that during a procedure three persons can be working at the same time, that is, on the operating part, and to its right at an instrument that is inserted through the proximal port 63, and to its left on an instrument that is inserted through the port 37, without disturbing one another.

In addition, FIG. 6 indicates in strong simplification that the port addition can be configured in a cross-sectional u-shaped profile for instance, rather than as two-plated. Moreover, FIG. 6 also shows another type of fastening, namely for instance by means of a screw or axis with an excenter piece, which can be inserted into the ends of the U-shaped post, and the excenter piece, when actuated by rotating the port addition, firmly grips the port addition onto the shaft or shaft port.

The functioning of the inventive endoscope is discussed hereafter with reference to endoscopically retrograde cholangio-pancreatography (ERCP).

During the phase of placing the mother duodenoscope 61 through the gullet, alimentary canal, and stomach into the duodenum, the shaft port must be closed because in the process about 100 to 110 cm of the endoscope shaft and thus the shaft port itself are found inside the patient. This can be achieved, for instance, as previously explained, by means of a cap 3 that can be inserted into the shaft port opening. Of course other forms of inner or outer coverings are also possible, such as a hollow probe inserted by the conventional proximal entry or port 63 on the operating part of the endoscope into the endoscope canal 9, which closes and insulates the shaft port from inside, or a locking sleeve whose opening is closed by rotating or sliding with respect to the shaft port opening, or else a thin-walled rubber or plastic hose (of the finger-cap type with cut-off tip) which is pushed onto the duodenoscope and unrolled over the shaft port opening.

After the placement the shaft port comes to rest outside the patient, since because of the stretching only a shaft length of less than 60 cm remains inside the patient. After removal of the covering and fastening of the port addition 37, the shaft port 1 then can be used as an entry for instruments. For this purpose the port addition 37 is insulated against the particular instrument, such as an inserted baby endoscope (for instance by means of a corresponding insulation in the port addition 37 or in its opening), to prevent blown-in air from escaping during the insertion as far as the duodenum and thus worsens the endoscopic viewing because of collapsing of the lumen of the duodenum. Of course it is also possible to introduce instruments directly into the shaft port without port addition 37, although for this purpose preferably a stronger air intake is needed to compensate for the air that possibly escapes by the shaft port.

The port addition 37 is inserted into the instrument canal or working canal 9 through the shaft port 1 and insulates the shaft port. On the outer end, in addition, a lateral support can be applied for the air insufflation. This support can possibly be connected to the normal air intake. Of course it is also possible to configure the port addition 37, in principle, as a very thin-walled (Teflon, etc.) hose, which is pushed into the working canal 9 essentially for insulation by the shaft port. Such a port addition 37 should preferably include an insulating cap as well as possibly lateral air supports that can be helpful for insufflation.

The fastening of the port addition 37 onto the shaft 15 can occur in a number of possible ways, such as an excenter piece (screw, lever, or the like) with which the port addition 37 is clamped. The port addition 37 is preferably equipped with a centering agent such as the previously described protuberance, centering pins, or the like and in this way is fastened on the shaft port in such a way that the insertion support 40 optimally flows into the instrument canal 9 and thus the baby endoscope can optimally be introduced with the least possible friction into the mother endoscope 61. Of course it is also possible to configure the port addition 37 as likewise capable of flapping upward, as previously described with reference to the flappable variant of the cap.

Such a baby endoscope has, for instance, an outer diameter of about 3.5 mm and thus includes about 0.5 mm free play with respect to the instrument canal (diameter about 4 mm) of the mother endoscope (for instance for insufflation). The outer surface of the baby endoscope is as slidable as possible (Teflon etc.) in order to offer low resistance to gliding. For bending in just one plane (up and down), the baby endoscope is preferably as stable against rotation as possible so that the absent bending planes can be reached through rotation. The baby endoscope has an instrumentation canal of at least 1 mm (preferably at least 1.4 mm), so that it is appropriate for electrohydraulic probes, biopsy tongs, stone baskets, etc. A good bending is guaranteed by the shaft port 37, even after insertion of the baby endoscope, so that the instrument achieves its clinical relevance on the basis of the good turnability alone.

Electrohydraulic lithotripsy can work with excellent results in cholangioscopy with a shortened baby. The probe adapted to the baby endoscope in its length is thus inserted with free play through the instrumentation canal of the baby endoscope. With the EHL probe inserted, a sufficient perfusion is preferably possible with the liquid required for the EHL.

Additional applications include rotation-stable catheters of about 120 cm length (for instance the catheter of the angiography type) with a diagonal nose for selective probing of the gall passages.

If an endoscope, in particular a duodenoscope, in addition to the inventive shaft port, includes a customary proximal port, the endoscope can in principle also be used as a routine endoscope, in particular a duodenoscope, for instance for conventional ERCP, so that two duodenoscopes are not required for the ERCP operation. The resulting cost savings (especially in smaller clinics with limited budget) can be advantageous.

1. An endoscope for mother-baby cholangioscopy, having a working canal that runs in or on a shaft of the endoscope and has an outlet opening distally on the endoscope head, characterized in that the working canal includes at least one insertion opening.

2. An endoscope according to claim 1, characterized in that the first insertion opening lies essentially in the center area of the shaft, at a distance of 60 to 70 cm from the distal end of the endoscope.

3. An endoscope according to claim 1, characterized in that the endoscope additionally includes another insertion opening in the area of the proximal end or on the endoscope housing.

4. An endoscope according to claim 3, characterized in that the first and the additional insertion openings lead to one and the same working canal, and the working canal extends at least to the other insertion opening.

5. An endoscope according to claim 1, characterized in that the first insertion opening is configured as capable of being closed and insulated by a stable covering.

6. An endoscope according to claim 5, characterized in that the covering on its underside includes a protruding area, which extends into the opening of the canal segment, where the protruding area is configured as flush with the canal interior, so that in the closed position there are no undesired resistances for an instrument introduced by a proximal port.

7. An endoscope according to claim 1, characterized in that an addition with an insertion support is positionable at the first insertion opening so that it is stable and removable to facilitate the insertion of instruments into the working canal.

8. An endoscope according to claim 7, characterized in that, with the addition applied, there arises an acute angle of 10 to 40 degrees between the longitudinal axis of the insertion support and the longitudinal axis of the working canal in the direction looking toward the proximal end of the endoscope.

9. An endoscope according to claim 1, characterized in that the area of the endoscope shaft, in which the first insertion opening is located, is configured as a rigid sleeve.

10. An endoscope according to claim 1, characterized in that the endoscope additionally includes a covering which serves to lock the first insertion opening and is stable and insulating.

11. An endoscope according to claim 1, characterized in that the endoscope additionally includes an addition, which is positioned, set off in profile, on the opposite side of a proximal additional insertion opening.

12-13. (canceled)

14. The endoscope of claim 1, wherein the endoscope is a duodenoscope.

15. An endoscope system comprising:
an endoscope according to claim 1;
a baby endoscope, which has a length of 80 to 140 cm, wherein the baby endoscope is inserted into the working canal of the endoscope.

16. The endoscope system of claim 15, wherein the baby endoscope has a length of 120 cm.

17. An endoscope arrangement for conducting a mirroring of the gall passages (cholangioscopy) comprising:
an endoscope for mother-baby cholangioscopy, having a working canal that runs in or on a shaft of the endoscope and has an outlet opening distally on the endoscope head, characterized in that the working canal includes at least one insertion opening;
an endoscope covering, wherein the covering serves to lock the first insertion opening and is stable and insulating;
an endoscope addition, wherein the addition is positioned, set off in profile, on the opposite side of a proximal additional insertion opening;
a baby endoscope which has a length of 80 to 140 cm wherein the baby endoscope can be inserted into the working canal of the endoscope.

18. The endoscope of claim 17, wherein the endoscope is a duodenoscope.

19. The endoscope system of claim 17, wherein the baby endoscope has a length of 120 cm.
